(19) United States
(12) Patent Application Publication
(10) Pub. No.: US 2010/0330523 A1
(43) Pub. Date: Dec. 30, 2010

(54) OPTICAL TIP FOR PHOTOSYNTHESIS

(75) Inventor: Jimmie Kert, (US)

Correspondence Address: Nordic Patent Services, Pilestraede 58, Copenhagen K, DK-1112, DK

(73) Assignee: CMS DENTAL APS, Copenhagen (DK)

(21) Appl. No.: 12/816,924

(22) Filed: Jun. 16, 2010

Related U.S. Application Data
(63) Continuation-in-part of application No. 12/257,050, filed on Oct. 23, 2008.
Application No. 12/257,050 is a continuation-in-part of application No. 11/694,475, filed on Mar. 30, 2007.

(51) Int. Cl. A61N 5/06

(52) U.S. Cl. 433/29; 433/215

(57) ABSTRACT

An optical tip for disinfecting a site of a periodontal pocket includes a first portion configured to be optically coupled to a light source, and a second portion configured to be introduced into the site of the periodontal pocket, where a distal end of the second portion is cylindrical in form and has a constant outer diameter. The second portion is configured to carry light from the light source to the distal end.

### CROSS-REFERENCE TO RELATED-APPLICATIONS

This application is related to, and is a continuation-in-part application of, U.S. patent application Ser. No. 12/257,050, filed on 23 Oct. 2008 and published as US Patent Pub. No. 2009/0047618, which is a continuation-in-part application of U.S. patent application Ser. No. 11/694,475, filed on 30 Mar. 2007 and published as US Patent Pub. No. 2008/0241785, commonly owned by the owner of the instant application, status pending, the disclosures of which are incorporated herein by reference in their entireties.

### FIELD

The aspects of the disclosed embodiments relate to the field of medical devices used by dental professionals. In particular, the aspects of the disclosed embodiments relates to an optical tip for photo activated disinfection in periodontal pockets.

### BACKGROUND

Periodontal disease is caused by a buildup of bacteria, mucous and microbes in the tissues surrounding the teeth. As the disease progresses, these tissues suffer deterioration causing them to separate from the roots of the teeth, creating periodontal pockets. Significant tooth bone loss can also occur. Treatment of deep periodontal pockets often involves scaling and root planning to mechanically remove the tartar and calculus from the teeth both above and underneath the gum line. In more serious cases of periodontitis, surgery is required in which gum tissue removal, flap reconstruction, bone reconstruction, and bone grafting may be necessary. After treatment procedures, further steps are taken to ensure that the sites are cleaned of all bacteria to promote site healing and prevent future buildup of calculus and infection. It is also thought that application of anti-bacterial medicaments may be an effective treatment of periodontal disease and may prevent excessive buildup of calculus.

A periodontal pocket is a dental term indicating the presence of an abnormally depended gingival sulcus as it contacts a tooth. The normal sulcular depth is approximately three millimeters or less. If the original depth is violated so much that the gingival fibers that initially attached the gingival tissue to the tooth are destroyed, the sulcus is termed a periodontal pocket. A periodontal pocket can also be described as a deep space between the gingival and the crown or root of a tooth.

There are currently many devices known in the art which employ the use of photosynthesis to kill bacteria and sterilize areas of the mouth affected by caries, wounds, periodontal pockets or surgical openings created by root canals and other procedures. In such a treatment method, a photo synthesizer solution such as toluidine blue dye in an aqueous solution is introduced to the site. On contact with the bacteria, the dye or other photo synthesizer conjugates with the bacteria. The photo synthesizer solution is absorbed by bacteria and releases O⁻ (singlet oxygen) when exposed to light with given wavelength which is specific to the absorption of each photo synthesizer solution. O⁻ is highly poisonous for bacteria. Thereafter, an optical fiber connected at one end to a laser unit containing a conventional laser is introduced to the area. The light generated by the laser is applied to the area to photo activate the photo synthesizer solution. The photo synthesizer solution releases O⁻ resulting in the death of the bacteria.

In known photo synthesizer devices, the tip or light guide is typically conical or tapered in order to allow the tip to reach into a narrow space such as a periodontal pocket. Other known tips include spherical shapes and blunt shapes to treat wider wounds and areas of the mouth. The tips mentioned above are generally configured to allow light to pass throughout the entire surface of the tip, thus allowing exposure over a wider portion of the target area. However, with the wider diffusion of light energy, a higher powered light source is required. Other, more targeted, tips are also known which include a small opening or aperture to allow the passage of light. Disadvantages of this type of tip or light guide are that the light energy is not evenly distributed across the target area, with a higher intensity being delivered to the area directly in front of the opening, and less energy being delivered to the surrounding areas.

It would be advantageous to have a photo synthesizer device and method for treating periodontal pockets which is adapted to treat a wide target area with a more equal distribution of energy to the area and requiring a less powerful light source.

### SUMMARY

As described herein, the exemplary embodiments overcome one or more of the above or other disadvantages known in the art.

One aspect of the exemplary embodiments relates to an optical tip for disinfecting a site of a periodontal pocket. In one embodiment, the optical tip includes a first portion configured to be optically coupled to a light source, and a second portion configured to be introduced into the site of the periodontal pocket, where a distal end of the second portion is cylindrical in form and has a constant outer diameter. The second portion is configured to carry light from the light source to the distal end.

Another aspect of the disclosed embodiments relates to an optical tip with a proximate end and a distal end. The proximate end is configured for direct coupling to a LED light source and has a receiver section for coupling to a lens of the LED light source. The receiver section includes a recess configured to receive the light emitted from the lens of the LED light source, a bottom surface and a sidewall portion. The bottom surface and sidewall portion are configured to collect light emitted from the lens and transmit the light to the distal end. The distal end includes a light emitting surface.

A further aspect of the disclosed embodiments relates to a method for disinfecting a periodontal pocket in an endodontic treatment. In one embodiment the method includes introducing a photo synthesizer into a site of the periodontal pocket, and introducing an optical tip into the site of the periodontal pocket. The optical tip includes a first portion configured to be optically coupled to a light source, and a second portion configured to be introduced into the site of the periodontal pocket. A distal end of the second portion is cylindrical in form and has a constant outer diameter. The second portion is configured to carry light from the light source to the distal end. The photo synthesizer is activated by applying light to the optical light tip.

These and other aspects and advantages of the exemplary embodiments will become apparent from the following detailed description considered in conjunction with the accompanying drawings. It is to be understood, however, that the drawings are designed solely for purposes of illustration and not as a definition of the limits of the invention, for which reference should be made to the appended claims. Moreover, the drawings are not necessarily drawn to scale and unless otherwise indicated, they are merely intended to conceptually illustrate the structures and procedures described herein. In addition, any suitable size, shape or type of elements or materials could be used.

### BRIEF DESCRIPTION OF THE DRAWINGS

In the following detailed portion of the disclosed embodiments, the invention will be explained in more detail with reference to the exemplary embodiments shown in the drawings, in which:

FIG. 1 illustrates an exemplary handset in accordance with an aspect of the disclosed embodiments;

FIG. 2 illustrates a exemplary light tip according to an embodiment of the invention;

FIG. 3 is an end view of the light tip shown in FIG. 2;

FIG. 4 is a perspective view of the exemplary light tip shown in FIG. 1; and

FIG. 5 illustrates the exemplary light tip of FIG. 1 attached to a handset device.

FIGS. 6-8 illustrates another exemplary light tip incorporating aspects of the disclosed embodiments.

FIG. 9 illustrates another exemplary light tip incorporating aspects of the disclosed embodiments.

### DETAILED DESCRIPTION OF THE DISCLOSED EMBODIMENTS

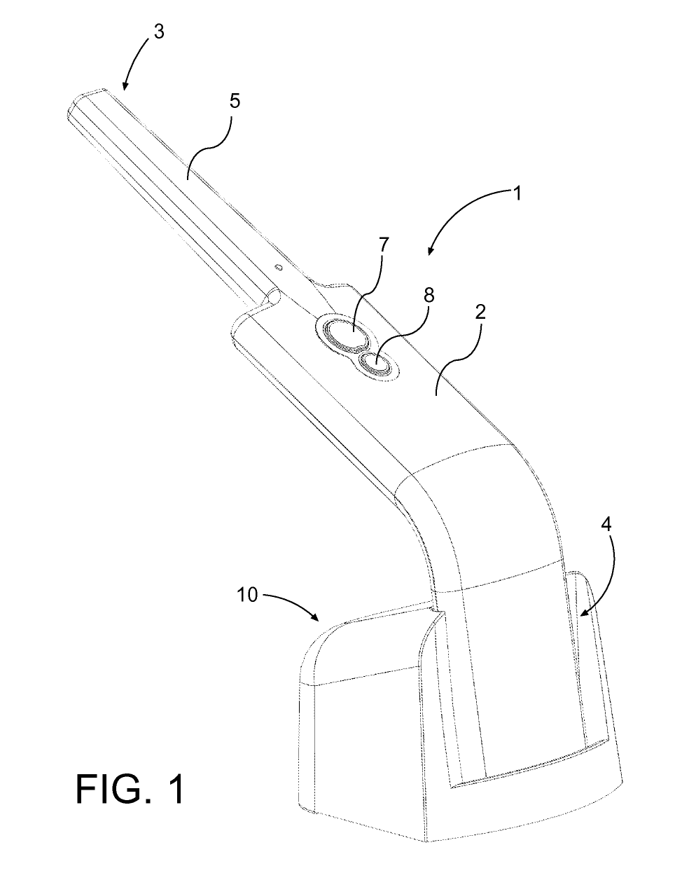

Reference will be made first to FIG. 1, which is an elevated view of an exemplary device for use in conjunction with the disclosed embodiments by a dental professional for disinfecting a periodontal pocket. The aspects of the disclosed embodiments are generally directed to an optical tip for a device used for photo activated disinfection, particularly with respect to periodontal pockets.

As is illustrated in FIG. 1, the device comprises a handset 1 and a base 10 on which the handset 1 can be placed when the handset 1 is not in use. In one embodiment, the handset 1 includes a housing 2 and has a distal end 3 and a proximal end 4. In one embodiment, the distal end 3 is provided with a user exchangeable cover 5. The user exchangeable cover 5 is secured to the housing 2 by a snap action or the like and can be made from a disposable material or from a material that can be auto-enclaved repetitively. An example of material that can be auto-enclaved repetitively is Grilamid(R) TR FE 5599.

The handset 1 includes an activation/deactivation button 7 and an activation/deactivation button 8 that are generally placed in the central portion of the housing 2. In alternate embodiments, the buttons 7 and 8 can be located on any suitable portion of the handset 1.

The handset 1 will generally also include a circuit board (not shown) for carrying the electronic components and a microprocessor to control the operations of the handset 1. The handset 1 can also include a suitable power supply, such as for example, a rechargeable battery of the lithium-ion type. In alternate embodiments, the handset 1 may be configured to be wired to a power source or may be operated using any suitable battery type. In one embodiment, the handset 1 may include a vibrator motor that includes an electric motor that is provided with an eccentric weight (mass) on the drive shaft.

In one embodiment, the handset 1 carries at its distal end 3 a light source 16 (shown in FIG. 5). In one embodiment the light source 16 comprises an LED light source. In alternate embodiments the light source 16 can comprise any suitable light source, such as for example, an incoherent or coherent laser source in place of or in addition to the light source 16.

The light source 16 is generally configured to emit light of wavelengths known in the art to activate photosynthetic solutions commonly used in endodontic application such as, for example, Toluidine Blue O (TBO) and Riboflavin (vitamin B2). In an alternate embodiment, the handset 1 may include an incoherent or coherent laser source in place of or in addition to the light source 16.

In one embodiment, the light source 16 may protrude from the user exchangeable cover 5 of the handset 1. In another embodiment, as depicted in FIG. 5, the light source 16 may be recessed into the housing 2 and the user exchangeable cover 5 may include a gap or recess in order that a tip may be optically coupled to the light source 16.

As shown in FIG. 1, in one embodiment, the handset 1 is electrically coupled at its proximal end 4 to the base 10 by suitable contacts for recharging the battery. The base 10 can be connected to suitable power mains.

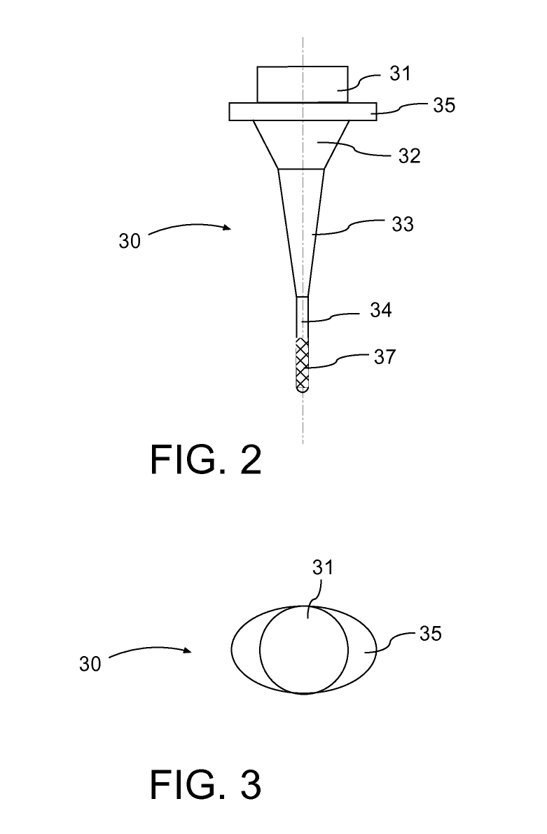

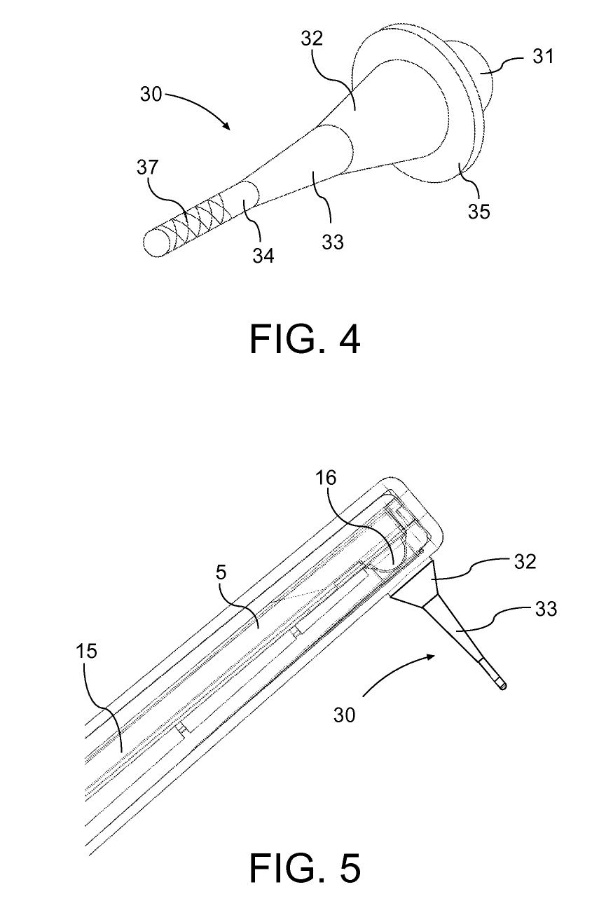

Referring also to FIGS. 2 and 4, the handset 1 is used in combination with an optical tip 30 that can be operably coupled to the handset 1. As shown in FIG. 2, the optical tip 30 generally comprises an optical fiber for use in a periodontal pocket of the tissues surrounding the tooth, to which access has been gained. The optical tip 30 is made from a material with suitable optical characteristics for light guides, such as various resins, plastics or combinations thereof. FIG. 5 illustrates one example of an optical tip 30 attached to a handset 1. In this example, an arm 15 carries at its distal end an LED 16 with a high optical output. The optical output of the LED is preferably above 1 W. A suitable LED is type nr. LE-015015R1G manufactured by LedEngin, Inc. This LED consumes approximately 15 W power and emits more than 1 W optical output, typically approximately 2 W (red light, peak frequency of approximately 625-640 nm). The spherical tip of the LED 16 can be used as a spherical coupling surface for coupling to the coupling portion 31 of the optical tip 30.

The light tip 30 generally includes a proximal portion 32, a middle portion 33 and a distal portion 34. The proximal portion 32 is substantially shaped like a conical frustum, and is slightly tapered to connect to the diameter of the middle portion 33. The middle portion 33 is generally tapered from the connection to the proximal portion 32 to the connection with the distal portion 34. The main function of the middle portion 33 is to connect the proximal portion 32 to the distal part 34. In one embodiment, the middle portion 33 is of a conical shape, is of lesser dimension than the proximal portion 31, and is tapered until it reaches the diameter of the distal portion 34. The middle portion 33 has a diameter that is preferably no greater than approximately 1-3 millimeters. In an alternate embodiment, the middle portion 33 is not tapered.

Although the middle portion 33 is described herein as functioning to connect the proximal portion 32 with the distal portion 34, in one embodiment, the proximal portion 34, middle portion 33 and distal portion 32 comprises a single or integrated piece. Alternatively, the light tip 30 can be made up of any suitable number of portions or parts. For example, one or more middle portions 33 may be used to extend or shorten an overall length of the light tip 10, where each subsequent middle portion 33 is suitably sized to provide a suitable taper, where tapering is a part of the middle portion 33, from the proximate portion 32 to the distal portion 34.

The distal portion 34 is shaped and dimensioned for introduction into the periodontal pocket. In one embodiment, the distal portion 34 is of cylindrical shape and has a lesser diameter than that of the middle portion 33, preferably no greater than approximately 1 millimeter. The dimensions described above generally enable the distal portion 34 to be substantially inserted into periodontal pockets of varying sizes and depths.

In one embodiment, referring to FIG. 4, for example, the distal portion 34 is not tapered. Rather, the distal portion 34 is substantially cylindrical, of a substantially constant diameter and generally straight. In one embodiment, the end of the distal portion 34 is substantially flat or slightly rounded. The distal portion generally comprises a light transmitting material or a light transmitting channel. In the embodiment where there is a light transmitting channel that can be hollow, in one embodiment, an inner diameter of the distal portion 34 is in the range of approximately 0.2 millimeters to 0.6 millimeters, while an outer diameter of the distal portion 34 is in the range of approximately 0.5 millimeters to 1 millimeters. In alternate embodiments, the outer diameter of the distal portion 34 can be any suitable diameter. The cylindrical form of the distal portion 34 forms a light spreading part of the tip 30. In one embodiment, the distal portion 34 comprises a light transmitting material, such as a clear plastic or acrylic.

As shown in FIGS. 2 & 4, the distal portion 34 of the tip 30 includes a roughened surface area 37. The roughened surface 37 is generally circumferential and runs along a portion of the length of the distal portion 34. In one embodiment, approximately ½ of the distal portion 34 is roughened to form the roughened surface 37. In a preferred embodiment, the roughened surface 37 extends along an approximately 3-5 millimeter length of the distal portion 34 of the tip 30. The roughened surface 37 is configured to allow the light from the light source 16 to diffuse from the light transmitting material of the distal portion 34. This roughened surface 37 allows for an even diffusing of the light from the light source 16, thus allowing for a more even application of light energy to the targeted area or periodontal pocket into which the tip 30 is introduced.

Referring to FIG. 2, in one embodiment, the light tip 30 also includes a coupling portion 31, which is generally configured to optically connect the optical tip 30 to the distal end 3 of the handset 1 near the light source 16 in order that light from the light source 16 can travel through the tip 30. In alternate embodiments, the coupling portion 31 of the optical tip 30 may also have the ability to couple with different handsets, or with a vibrator motor of a handset.

In a preferred embodiment, as depicted in FIG. 5, the coupling portion 31 may have a concave shape in order to couple securely directly to the convex surface of the light source 16. Since the light source 16 is recessed in the housing 2, the user exchangeable cover 5 is provided with a gap or recess into which the coupling portion 31 may be received. In an alternate embodiment, the distal end 3 or the user exchangeable cover 5 may include a convex coupling surface separate from, or in addition to the surface of the light source 16. However, in a further embodiment the arrangement can be reversed with a concave coupling surface on the distal end 3 of the handset 1 and a convex coupling surface on the coupling portion 31 of the optical tip 30. In the exemplary embodiment depicted in FIG. 2, the coupling portion 31 of the optical tip 30 is a cylindrical projection for optical connection to a convex surface of a light source 16 or coupling surface of the handset 1.

The optical coupling between the concave and convex surfaces allows for light emitted by the light source 16 to be guided into the optical tip 30. The convex surface can mate with the concave surface over a range of angles in a fashion similar to that of a ball joint, thereby reducing the need to align the handset 1 with the optical tip 30 and thus facilitating the use in the relatively small periodontal pocket or other suitable oral cavity.

In one embodiment as shown in FIG. 2, the optical tip 30 includes a collar 35. The collar 35 is generally configured to create a barrier or seal with the user exchangeable housing 5 when the coupling portion 31 is optically connected to the handset 1. This collar or barrier 35 generally prevents water droplets or other particles from penetrating the housing 2 of the handset 1. As shown in FIG. 3, the collar 35 is generally configured to be larger in circumference than a diameter of the coupling portion 31. Although the collar 35 is shown in FIG. 3 in the form of an ellipse, in alternate embodiments the collar 35 can comprise any suitable geometric shape, such as for example a circle or square.

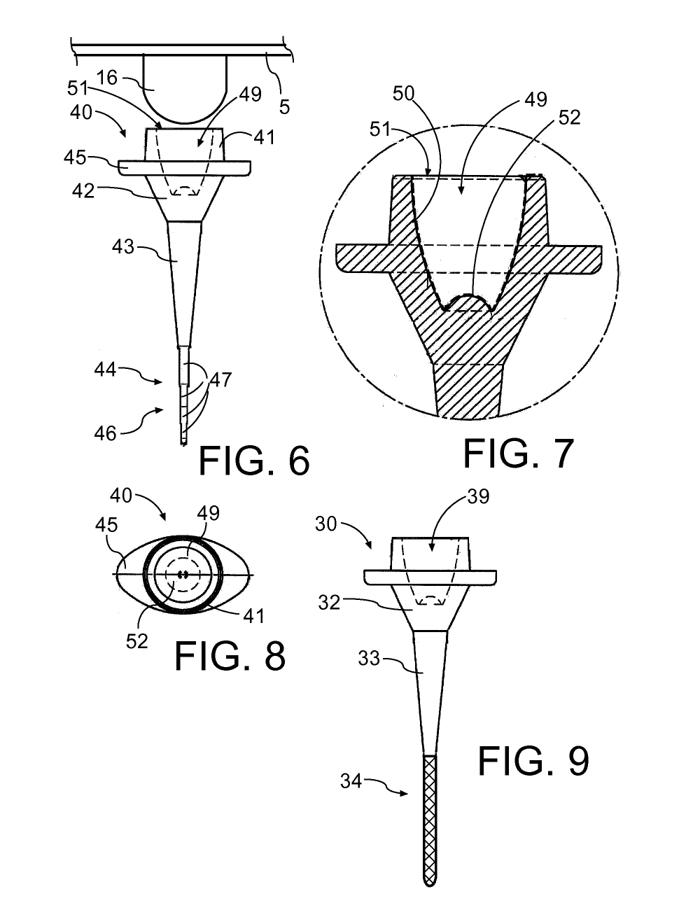

Another embodiment of an exemplary optical tip 40 incorporating aspects of the present disclosure is illustrated in FIGS. 6-8. The optical tip 40 generally includes a proximal portion 42, a middle portion 43 and a distal portion 44. In this example, the proximal portion 42 is substantially shaped like a conical frustum, and is slightly tapered to connect to the diameter of the middle portion 43. The middle portion 43 is generally tapered from the connection with the proximal portion 42 to the connection with the distal portion 44. In one embodiment, the proximal portion 42 and the middle portion 43 can form a single piece. The main function of the middle portion 43 is to connect the proximal portion 42 to the distal part 44. In one embodiment, the middle portion 43 is of a conical shape, is of lesser dimension than the proximal portion 42, and is tapered until it reaches the diameter of the distal portion 44. In one embodiment, the middle portion 43 has a diameter that is preferably no greater than approximately 1-3 millimeters. In an alternate embodiment, the middle portion 43 is not tapered.

The distal part 44 is generally shaped and dimensioned for introduction into the root canal or other periodontal area or pocket. In one embodiment, the distal part 44 tapers stepwise towards the distal end of the optical tip 40 to form a stepwise tapered or tapering portion 46. The steps in the stepwise tapering portion 46 form light emitting surfaces. In one embodiment, the stepwise tapering portion 46 includes a plurality of substantially cylindrical sections 47 between the steps. Since the steps are distributed over the length of the tapered portion 46, the light entering the optical tip 40 is distributed along the length of the periodontal pocket or area, such as a root canal, into which the optical tip 40 is introduced.

As shown in FIGS. 6-8, in this embodiment, the proximal portion 42 includes a receiver or cavity 49 for optically coupling the LED 16 to the optical tip 40. The receiver 49 generally includes an opening or recess 51 at the top surface and a substantially curved or convex bottom surface 52. The receiver 49 is generally configured to collect the light transmitted through a lens of the light source LED 16. In this embodiment, the curved bottom surface 52 bulges toward the opening 51 and the light source LED 16. The curved bottom surface 52 allows the light from the LED 16 to be collected and directed into the distal part 44 of the tip 40. Since the LED 16 is generally spherical in shape, the LED 16 scatters and spreads the light. If the bottom surface of receiver 49 where the light from the LED 16 enters the tip 40 is a substantially flat surface, at least a portion of the light transmitted from the LED 16 will reflect from the bottom surface and not propagate to the distal part 44 of the tip 40. A substantially convex bottom surface 52 allows the light from the LED 16 to be collected and directed into the distal part 44 of the tip 40, resulting in a greater portion of the light from the LED 16 being transmitted to and from the distal part 44. It is estimated that the use of a substantially curved bottom surface 52 as is described herein can result in improvements of approximately 15% in the amount of light delivered to the distal part 44 of the tip 40, in comparison to tips that do not have such a substantially curved bottom surface, enabling or allowing more light to be introduced into the periodontal pocket or space, such as a root canal, for example, entered by the tip 40.

In one embodiment, as is shown in FIGS. 6-8, the sidewalls 50 of the receiver section 49 are curved. Generally, the recess is substantially rotation symmetric. In the example shown in FIGS. 6-8, the sidewalls 50 curve inwards, with the opening 51 of the receiver section 49 having a diameter that is larger than the diameter of the bottom surface 52. The generally curved shape of the receiver section 49 is advantageous for production, particularly where injection moulding is utilized.

FIG. 8 illustrates a top down look into the optical tip 40. In one embodiment as shown in FIG. 8, the optical tip 40 includes a collar 45. The collar 45 is generally configured to create a barrier or seal with the user exchangeable housing 5 when the optical tip 40 is optically connected to the handset 1. This collar or barrier 45 generally prevents water droplets or other particles from penetrating the housing 2 of the handset 1. As shown in FIG. 8, the collar 45 is generally configured to be larger in circumference than a diameter of the coupling portion 41. Although the collar 45 is shown in FIG. 8 in the form of an ellipse, in alternate embodiments the collar 45 can comprise any suitable geometric shape, such as for example a circle or square.

FIG. 9 illustrates one embodiment of a tip 30, where the proximal portion 32 includes a receiver 39. The receiver 39 shown in FIG. 9 is substantially similar to the receiver 49 described with respect to FIGS. 6-8.

While the invention has been described with reference to the disinfection of periodontal pockets, it is understood that the applications of the invention are not so limited. The optical tip as described in the preferred embodiments may, in addition, be used in the treatment and disinfection of oral cavities, caries, root canals, and other such applications.

Thus, while there have been shown, described and pointed out, fundamental novel features of the invention as applied to the exemplary embodiments thereof, it will be understood that various omissions and substitutions and changes in the form and details of devices illustrated, and in their operation, may be made by those skilled in the art without departing from the spirit of the invention. Moreover, it is expressly intended that all combinations of those elements and/or method steps, which perform substantially the same function in substantially the same way to achieve the same results, are within the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. An optical tip for disinfecting a site of a periodontal pocket comprising:
a first portion configured to be optically coupled to a light source; and
a second portion configured to be introduced into the site of the periodontal pocket, where a distal end of the second portion is cylindrical in form and comprises a constant outer diameter, the second portion being configured to carry light from the light source to the distal end.

2. The optical tip according to claim 1 wherein the distal end of the second portion further comprises a roughened surface extending around a circumference of the outer diameter.

3. The optical tip according to claim 2 wherein the roughened surface is configured to diffuse the light from the light source into the periodontal pocket.

4. The optical tip according to claim 3 wherein a type of the roughened surface is configured to diffuse light having a specific wavelength.

5. The optical tip according to claim 1 wherein the roughened surface extends along approximately one-half of a length of the distal end.

6. The optical tip according to claim 1 wherein the roughened surface extends for a distance in the range of approximately 3 mm to 5 mm from an end of the second portion being introduced into the periodontal pocket.

7. The optical tip according to claim 1 wherein an end of the distal end is substantially flat or slightly rounded.

8. The optical tip according to claim 7 further comprising that the distal end is not tapered towards an end of the second portion being introduced into the periodontal pocket.

9. The optical tip according to claim 1 wherein the second portion comprises a middle portion and a distal portion, the middle portion being coupled to the first portion, wherein the middle portion is cylindrical in shape tapering from the first portion towards the distal portion.

10. The optical tip according to claim 1 further comprising that the distal portion has a constant outer diameter along its length.

11. The optical tip according to claim 10 wherein the distal portion further comprises a roughened surface extending around a circumference of the distal portion.

12. The optical tip according to claim 11 wherein the roughened surface extends along a portion of a length of the distal end that is introduced into the periodontal cavity.

13. The optical tip according to claim 1, wherein said optical tip is an optical fiber.

14. The optical tip according to claim 1 wherein said optical tip is comprised of a light transmitting material.

15. The optical tip according to claim 1, wherein the first portion further comprises a cylindrical portion for mechanical connection to a hand piece including the light source.

16. The optical tip according to claim 1 wherein an outer diameter of the distal end is in the range of approximately 1 mm and 3 mm.

17. An optical tip with a proximate end and a distal end, said proximate end being configured for direct coupling to a LED light source and having a receiver section for coupling to a lens of the LED light source, wherein the receiver section comprises:
a recess configured to receive the light emitted from the lens of the LED light source;
a bottom surface and a sidewall portion, the bottom surface and sidewall portion being configured to collect light emitted from the lens and transmit the light to the distal end; and
wherein the distal end comprises a light emitting surface.

18. The optical tip according to claim 17, wherein the bottom surface is a substantially curved surface that bulges towards the lens of the LED light source.

19. The optical tip according to claim 17, where the sidewall portion is substantially curved from a top to the bottom surface.

20. The optical tip according to claim 17, wherein the recess is substantially rotation symmetric and wherein an opening of the recess has a diameter that is larger than a diameter of the bottom surface.

21. The optical tip according to claim 17 wherein the distal end of the tip comprises a stepwise taper that form light emitting surfaces.

22. The optical tip according to claim 17, wherein the distal end of the tip comprises a roughened surface extending around a circumference of an outer diameter of the distal end.

23. A method for disinfecting a periodontal pocket in an endodontic treatment, the method comprising:
introducing a photosynthesizer into a site of the periodontal pocket;
introducing an optical tip into the site of the periodontal pocket, wherein the optical tip comprises:
a first portion configured to be optically coupled to a light source; and
a second portion configured to be introduced into the site of the periodontal pocket, where a distal end of the second portion is cylindrical in form and comprises a constant outer diameter, the second portion being configured to carry light from the light source to the distal end; and
activating the photosynthesizer by applying light to the optical light tip.

24. The method of claim 23 wherein the distal end of the second portion further comprises a roughened surface extending around a circumference of the outer diameter.

25. The method of claim 24 further comprising diffusing the light from the light source into the periodontal pocket through the roughened surface.

26. The method of claim 25 wherein the distal end of the second portion that is introduced into the site of the periodontal pocket has a constant outer diameter along its length.

27. The method of claim 23 wherein the wherein the first portion comprises a receiver section for coupling to the light source, the receiver section having a bottom surface and a sidewall surface configured to collect light from the light source and transmit the light to the second portion, wherein the bottom surface is a substantially curved surface that bulges towards the light source.

28. The method of claim 27, where the sidewall surface is substantially curved and an opening of the receiver section has a diameter that is larger than a diameter of the bottom surface.
